(19) United States
(12) Patent Application Publication
(10) Pub. No.: US 2023/0086239 A1
(43) Pub. Date: Mar. 23, 2023

(54) SYSTEMS, METHODS, AND DEVICES FOR REMOTELY DEPLOYED DIAGNOSTIC TESTING

(71) Applicant: EMED Labs, LLC, Miami, FL (US)

(72) Inventors: Mitchell MORRIS, Dallas, TX (US); Michael W. FERRO, Jr., Palm Beach, FL (US); Sam MILLER, Hollywood, FL (US); Adam Charles Carlson, Miami, FL (US); Zachary Carl Nienstedt, Wilton Manors, FL (US)

(21) Appl. No.: 17/932,501

(22) Filed: Sep. 15, 2022

Related U.S. Application Data
(60) Provisional application No. 63/261,319, filed on Sep. 17, 2021.
(60) Provisional application No. 63/261,639, filed on Sep. 24, 2021.
(60) Provisional application No. 63/263,225, filed on Oct. 28, 2021.

(51) Int. Cl. G16H 40/67; G06Q 30/04

(52) U.S. Cl. CPC G16H 40/67; G06Q 30/04

(57) ABSTRACT

This disclosure relates to remote medical testing. Embodiments of this disclosure relate to systems, methods, and devices for providing remote medical testing services in developing regions or remote locations. Some embodiments include cost-shifting to enable users of remote medical testing services to shift data costs to a third party. Some embodiments include data transfer protocols for transmitting testing data to a remote server. Some embodiments include remotely-deployable testing apparatuses.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

This application claims the benefit under 35 U.S.C. § 119(e) to U.S. Provisional Patent Application No. 63/261319, titled “COST-SHIFTED REMOTE TESTING SESSIONS,” filed Sep. 17, 2021, U.S. Provisional Patent Application No. 63/261639, titled “DATA TRANSFER PROTOCOLS FOR REMOTE TESTING SESSIONS,” filed Sep. 24, 2021, and U.S. Provisional Application No. 63/263225, titled “SYSTEMS, METHODS, AND DEVICES FOR REMOTELY-DEPLOYABLE DIAGNOSTIC TESTING,” filed Oct. 28, 2021, each of which is incorporated herein by reference in their entirety and for all purposes.

### BACKGROUND

### Field

The present application is directed to remote testing sessions. Some embodiments are directed to reducing cost to the test user. Some embodiments are directed to data transfer protocols. Some embodiments are directed to remotely-deployable testing apparatuses.

### Description of the Related Art

The approaches described in this section are approaches that could be pursued, but not necessarily approaches that have been previously conceived or pursued. Thus, unless otherwise indicated, it should not be assumed that any of the material described in this section qualifies as prior art merely by virtue of its inclusion in this section.

Use of telehealth to deliver healthcare services has grown consistently over the last several decades and has experienced very rapid growth in the last several years. Telehealth can include the distribution of health-related services and information via electronic information and telecommunication technologies. Telehealth can allow for long-distance patient and health provider contact, care, advice, reminders, education, intervention, monitoring, and remote admissions. Often, telehealth can involve the use of a user or patient's personal computing device (also referred to herein as a user device), such as a smartphone, tablet, laptop, personal computer, or other type of personal computing device. For example, a user or patient can interact with a remotely-located medical care provider using live video, audio, or text-based chat through the personal device. Generally, such communication occurs over a network, such as a cellular or internet network.

### SUMMARY

This application describes systems, methods, and devices for reducing data usage costs for testing users. These systems, methods, and devices may allow more users to take advantage of remote testing and may improve the user experience by reducing data usage charges that are unexpected or unaffordable to the user. In some embodiments, data usage charges or other fees may be shifted from the testing user to the testing provider. Some embodiments of this application provide data transfer protocols that can be used to increase the availability and usefulness of telehealth in circumstances where communication networks are unavailable or limited. In some circumstances, users may be located in remote areas with limited infrastructure, which may create logistical problems in delivering tests, collecting test samples, and delivering results and/or treatment. Users may also lack access to electronic devices needed for taking remote medical tests such as smartphones, tablets, laptops, personal computers, or other types of personal devices. Additionally, network connectivity may be poor or even non-existent in some areas, creating difficulties with transmitting testing information to a remote testing service for storage, review by proctors, or other uses. Thus, some embodiments provide for remotely-deployable testing apparatuses.

For purposes of this summary, certain aspects, advantages, and novel features are described herein. It is to be understood that not necessarily all such advantages may be achieved in accordance with any particular embodiment. Thus, for example, those skilled in the art will recognize that the present disclosure may be embodied or carried out in a manner that achieves one or more advantages taught herein without necessarily achieving other advantages as may be taught or suggested herein.

In some aspects, the techniques described herein relate to a method for redirecting billing by a wireless data plan provider to a medical diagnostic testing service, the method including: exchanging data between a testing platform and a user device; generating a message requesting that charges associated with a remote medical diagnostic test be billed to the medical diagnostic testing service; and transmitting the message from the testing platform to the wireless data plan provider.

In some aspects, the techniques described herein relate to a method, wherein the message is generated by the testing platform.

In some aspects, the techniques described herein relate to a method, wherein the message is generated by the user device.

In some aspects, the techniques described herein relate to a method, wherein the message is transmitted to the wireless data plan provider by the testing platform.

In some aspects, the techniques described herein relate to a method, wherein the message is transmitted to the wireless data plan provider by the user device.

In some aspects, the techniques described herein relate to a method, wherein the message includes an amount of data used by the remote medical diagnostic test.

In some aspects, the techniques described herein relate to a method, wherein the message includes an estimated amount of data used by the remote medical diagnostic test.

In some aspects, the techniques described herein relate to a method, wherein the message includes a start time of the remote medical diagnostic test and an end time of the remote medical diagnostic test.

In some aspects, the techniques described herein relate to a method, wherein the message includes an amount of data used by a plurality of remote medical diagnostic tests.

In some aspects, the techniques described herein relate to a method, wherein the message includes a maximum data usage amount.

In some aspects, the techniques described herein relate to a method, wherein the message includes an estimate of an amount of data used by a plurality of remote medical diagnostic tests.

In some aspects, the techniques described herein relate to a method, wherein the message includes a plurality of start times and a plurality of end times of a plurality of remote medical diagnostic tests.

In some aspects, the techniques described herein relate to a system for cost-shifting data used for a remote medical diagnostic test including: a non-transitory computer-readable medium with instructions encoded thereon; and one or more processors configured to execute the instructions to cause the system to: receive, from a user device, identifying information indicative of a wireless data plan associated with the user device; provide the remote medical diagnostic test to a user via a user device; receive, from the user device, data related to the medical diagnostic test; determine an indication of an amount of data used for the remote medical diagnostic test; generate a message to request cost-shifting of the amount of data from the user to a testing platform provider; and send the message to a provider of the wireless data plan.

In some aspects, the techniques described herein relate to a system, wherein the message includes the indication of the amount of data used for the remote medical diagnostic test.

In some aspects, the techniques described herein relate to a system, wherein the message includes identifying information indicative of the wireless data plan.

In some aspects, the techniques described herein relate to a system, wherein the indication of the amount of data used for the remote medical diagnostic test includes an amount of data used for the remote medical diagnostic test.

In some aspects, the techniques described herein relate to a system, wherein the indication of the amount of data used for the remote medical diagnostic test includes an estimated amount of data used for the remote medical diagnostic test.

In some aspects, the techniques described herein relate to a system, wherein the indication of the amount of data used for the remote medical diagnostic test includes a start time and a stop time of the remote medical diagnostic test.

In some aspects, the techniques described herein relate to a system, wherein message includes a maximum data usage amount.

In some aspects, the techniques described herein relate to a system, wherein the non-transitory computer-readable medium has instructions that encoded thereon that, when executed by the one or more processors, cause the system to: determine, based on the identifying information, that cost-shifting is not available for the medical diagnostic test; and provide, to the user, an option to select a type of test experience; receive, from the user, a selection of the type of test experience, wherein to providing the remote medical diagnostic test to the user is based at least in part on the selection of the type of test experience.

In some aspects, the techniques described herein relate to a method for controlling data charges to a user of a medical diagnostic testing service, the method including: receiving information related to a user's wireless data plan provider associated with the user; determining if the medical diagnostic testing service can redirect billing by the wireless data plan provider from the user to the medical diagnostic testing service; and based on the determination, providing a different testing experience to the user.

In some aspects, the techniques described herein relate to a method, wherein the testing experience is modified to reduce an amount of data transferred between a user device and the medical diagnostic testing service.

In some aspects, the techniques described herein relate to a method, wherein the information related to a user's wireless data plan provider is provided manually by the user.

In some aspects, the techniques described herein relate to a method, wherein the information related to the user's wireless data plan provider is provided automatically by the wireless data plan provider.

In some aspects, the techniques described herein relate to a method, wherein the information related to the user's wireless data plan provider is provided automatically by a user device.

In some aspects, the techniques described herein relate to a remotely-deployable testing apparatus including: a lower outer container portion; a lower inner container portion; an upper outer container portion; an upper inner container portion; one or more fiducial markers; one or more hardware components configured for coupling of the remotely-deployable testing apparatus to a delivery vehicle; and two or more legs coupled to the lower container portion.

In some aspects, the techniques described herein relate to a remotely-deployable testing apparatus, further including: a computing device, wherein the computing device includes a camera, and wherein the computing device is mounted to the upper inner container portion.

In some aspects, the techniques described herein relate to a remotely-deployable testing apparatus, further including: one or more solar panels; and a charge controller, wherein the charge controller is configured to regulate a voltage, a current, or both provided by the one or more solar panels.

In some aspects, the techniques described herein relate to a remotely-deployable testing apparatus, further including an ultraviolet sterilization compartment.

In some aspects, the techniques described herein relate to a remotely-deployable testing apparatus, further including a receptacle to receive medical waste.

In some aspects, the techniques described herein relate to a remotely-deployable testing apparatus, further including a satellite phone.

In some aspects, the techniques described herein relate to a remotely-deployable testing apparatus, further including wireless networking hardware configured to provide a wireless local area network.

In some aspects, the techniques described herein relate to a remotely-deployable testing apparatus, further including one or more diagnostic tests.

In some aspects, the techniques described herein relate to a remotely-deployable testing apparatus, further including: one or more writable radio frequency identification tags; and an RFID writer, wherein the RFID writer is configured to store one or more diagnostic test results on the one or more writable radio frequency identification tags.

In some aspects, the techniques described herein relate to a remotely-deployable testing apparatus, further including a printer configured to print one or more printable codes, wherein the one or more printable codes include one or more diagnostic test results.

In some aspects, the techniques described herein relate to a remotely-deployable testing apparatus, wherein the computing device is configured to store data relating to one or more diagnostic testing sessions.

All of the embodiments described herein are intended to be within the scope of this disclosure. These and other embodiments will be readily apparent to those skilled in the art from the following detailed description, having reference to the attached figures. The invention is not intended to be limited to any particular disclosed embodiment or embodiments.

### BRIEF DESCRIPTION OF THE DRAWINGS

These and other features, aspects, and advantages of the present application are described with reference to drawings of certain embodiments, which are intended to illustrate, but not to limit, the present disclosure. It is to be understood that the attached drawings are for the purpose of illustrating concepts disclosed in the present application and may not be to scale.

FIG. 1 is a schematic diagram illustrating an embodiment of a testing and billing adjustment request process.

FIG. 2 is an example flowchart showing a selection of different testing experiences according to an embodiment.

FIG. 3 is a block diagram depicting an embodiment of a protocol or method for data transfer that can be used to provide remote testing.

FIG. 4 is a block diagram depicting an embodiment of a protocol or method for data transfer that can be used to provide remote testing using a plurality of testing sessions.

FIG. 5 is a block diagram depicting an embodiment of a protocol or method for data transfer that can be used to provide remote testing using a plurality of testing sessions, wherein data can be prioritized for upload.

FIG. 6A shows an example embodiment of a remotely-deployable testing apparatus in a closed state.

FIG. 6B is an example embodiment of a remotely-deployable testing apparatus in an open state.

FIG. 7 illustrates an embodiment of a computer system that can be configured to perform one or more of the methods or processes described herein.

### DETAILED DESCRIPTION

Although several embodiments, examples, and illustrations are disclosed below, it will be understood by those of ordinary skill in the art that the inventions described herein extend beyond the specifically disclosed embodiments, examples, and illustrations and includes other uses of the inventions and obvious modifications and equivalents thereof. Embodiments of the inventions are described with reference to the accompanying figures, wherein like numerals refer to like elements throughout. The terminology used in the description presented herein is not intended to be interpreted in any limited or restrictive manner simply because it is being used in conjunction with a detailed description of certain specific embodiments of the inventions. In addition, embodiments of the inventions can comprise several novel features and no single feature is solely responsible for its desirable attributes or is essential to practicing the inventions herein described.

Remote or at-home healthcare testing and diagnostics can solve or alleviate some problems associated with in-person testing. For example, health insurance may not be required, travel to a testing site is avoided, and tests can be completed at a testing user's convenience. However, remote or at-home testing introduces various additional logistical and technical issues, such as guaranteeing timely test delivery to a testing user, providing test delivery from a testing user to an appropriate lab, ensuring proper sample collection, ensuring test verification and integrity, providing test result reporting to appropriate authorities and medical providers, and connecting testing users with medical providers, who are needed to provide guidance and/or oversight of the testing procedures remotely.

While remote or at-home health care testing offers many benefits, some users may only be able to access testing using data services that charge based on data usage and/or may be located in remote areas with limited physical and/or technological infrastructure. For example, users may only have access to a cellular data plan or other wireless data plan that incurs fees based on actual data usage or that imposes limits or caps on the amount of data a user may use. For some testing users, especially, for example, those in poorer or developing countries or in more remote areas, data usage charges may present a significant barrier to the use of remote testing. In some cases, users or patients may not have a personal device that they can use for remote medical testing, thus there can be a need to provide a computing device that can be used for remote medical testing. In some cases, technological infrastructure may be insufficient for sending data to a remote server, and a computing device, test samples, and so forth can be retrieved from a remote location.

People in developing countries often have limited access to medical care. These areas also often have limited internet connectivity, and users may rely on wireless data plans that have small, fixed limits on data usage or that incur fees based on data usage. People in these remote areas could benefit from greater access to medical testing, but poor connectivity and high costs associated with using wireless data may prevent potential users from taking advantage of remotely administered medical testing.

Some users may have only limited access to a suitable communication network. Limited access to a suitable communication network can be especially prevalent in more remote or less developed areas of the world. Often, these same remote and less areas are otherwise ideal beneficiaries of remote or at-home health care testing, as people who live in such areas may also have limited access to in-person healthcare or doctors. Insufficient network access can thus present a significant barrier to telehealth for such users.

Some embodiments of this application provide data transfer protocols that can be used to increase the availability and usefulness of telehealth in circumstances where communication networks are unavailable or limited. For example, various embodiments described herein relate to systems, methods, and devices for collecting data during a testing session and, while a network connection is limited or unavailable, caching said data locally. The cached data can be saved for transmission to a testing platform once a suitable network connection becomes available. In some embodiments, the devices, systems, and methods described herein may maintain a cache of data (e.g., testing session data) locally until a suitable network connection is available and established. At that point, the data may be transmitted to a testing platform for review, analysis, storage, etc.

The present disclosure provides systems, methods, and devices that can reduce or shift the cost for users of remote medical diagnostic testing by redirecting charges for data usage from the user to a medical diagnostic testing service, thus avoiding charges to the user and/or usage of the user's data allowance. Medical diagnostic testing services may enter into arrangements with wireless data plan providers to exchange information about data used for medical diagnostic testing that should be billed to the medical diagnostic testing service. Alternatively or additionally, in some embodiments where the medical diagnostic testing service may be unable to redirect billing, the user may instead be offered a testing experience with lower data requirements, or the user may be reimbursed directly for the data used by the test.

### Cost Shifting

As mentioned briefly above and as will now be explained in more detail, this application describes systems, methods, and devices for reducing data usage costs for users of remotely administered medical tests. Such systems, methods, and devices can, in some embodiments, involve cost-shifting from a user to a testing provider.

In some embodiments, the data exchanged between a user device and testing platform during a medical diagnostic test may include, for example, video of the testing user, audio of the testing user, video of a proctor or guide, audio of a proctor or guide, text-based instructions, augmented reality-based guidance, telemetry data such as device specifications or network capabilities, or other data.

In some embodiments, prior to, during, or after a testing session, the user device used during the testing session may generate a message requesting that data charges incurred during the test be billed to the medical diagnostic testing service. In some embodiments, the testing platform may generate a message requesting that data charges incurred during the test be billed to the medical diagnostic testing service. In some embodiments, the message includes the amount of data used for the medical test (e.g., as determined by the user device, the network through which the user device communicates with the testing platform, or the testing platform itself).

In some embodiments, the message includes an estimate of the data used for the medical test (e.g., rather than a direct measure of the data used). In some embodiments, the message includes a timestamp indicating when the test started and a timestamp indicating when the test ended. In some embodiments, the message may include, for example, a detailed list of data exchanges, actual costs incurred, calculated costs incurred, estimated costs incurred, etc. In some embodiments, timestamps (e.g., start times and/or stop times) may be used in combination with other data to estimate the amount of data used for the medical test. In some embodiments, an estimated amount of data can be the same as an amount of data used between a start time and a stop time. In some embodiments, costs can be shifted up to a maximum amount of data, and the message can include a maximum data usage amount. For example, if a testing session uses more than a maximum amount of data, the costs associated with the data may only be shifted up to the maximum amount. This can occur if, for example, data usage is estimated based on timestamps and the user was using data for other purposes at the same time as they were taking the remote test, such as watching videos or playing music.

In some embodiments, the user device may send the message to the wireless data plan provider. In some embodiments, the testing platform may send the message to the wireless data plan provider. For example, the testing platform can provide a test to a user, can receive test data from the user, and can subsequently provide, to the wireless data plan provider, a message indicative of the amount of data used. In some embodiments, the message may be communicated to the wireless data plan provider using an application programming interface (API). In some embodiments, the request is sent immediately or very soon after the test is concluded. In some embodiments, the request is sent on a schedule, such as hourly, daily, weekly, or monthly, or another schedule. In some embodiments, a message is sent at the beginning of the test and at the end of the test. In some embodiments, the request may relate to data used for more than one medical test. In some embodiments, a message is sent for each exchange of data. In some embodiments, a message is sent for more than one exchange of data. In some embodiments, the request may relate to data used for a single medical test. In some embodiments, the request may relate to data used for more than one medical test taken from a single user device. In some embodiments, the request may relate to data used for more than one test taken from more than one user device. For example, in some embodiments, a single request may relate to data used by multiple subscribers to a single wireless data plan provider.

In some embodiments, prior to, during, or after a testing session, the user may be asked to provide identifying information about the user's wireless data plan, such as the provider and the user's phone number, account number, or other information that may be used to identify the user or the account. In some embodiments, information about the user's wireless data plan may be determined automatically by querying the user's device, the wireless network, or both. In some embodiments, some or all of the collected identifying information can be included in a message to the wireless data plan provider.

In some embodiments, the testing platform and/or an application on the user device may have information relating to which wireless data plan providers will make billing adjustments. In some embodiments, if the wireless data plan provider may not make a billing adjustment, the user may be given a choice of discontinuing the test, continuing with the test using a regular experience which may include, for example, audio, video, augmented reality, and/or the like, or continuing with the test using a reduced data experience. In some embodiments, a reduced data test experience may use, for example, text-based instructions or pictures instead of, for example, video or audio. In some embodiments, the user may upload only critical information to the testing platform, such as video or photos of critical steps. In some embodiments, the user may communicate with a proctor or guide using text. In some embodiments, the user may communicate with a proctor or guide using, for example, video and/or audio.

In some embodiments, the wireless network provider may adjust the user's data usage immediately after receiving a message requesting a billing adjustment. In some embodiments, the wireless network provider may adjust the user's data usage at another time such as, for example, at the end of the user's billing cycle. In some embodiments, the user's data provider may bill the testing platform directly for charges incurred during a testing session. In some embodiments, the testing provider may preemptively pay the user's data provider for charges incurred during the test prior to the data provider billing the user for such charges. This can, in some embodiments, avoid such charges appearing on a user's bill at all and/or having such charges or data usage affect the user's data limit.

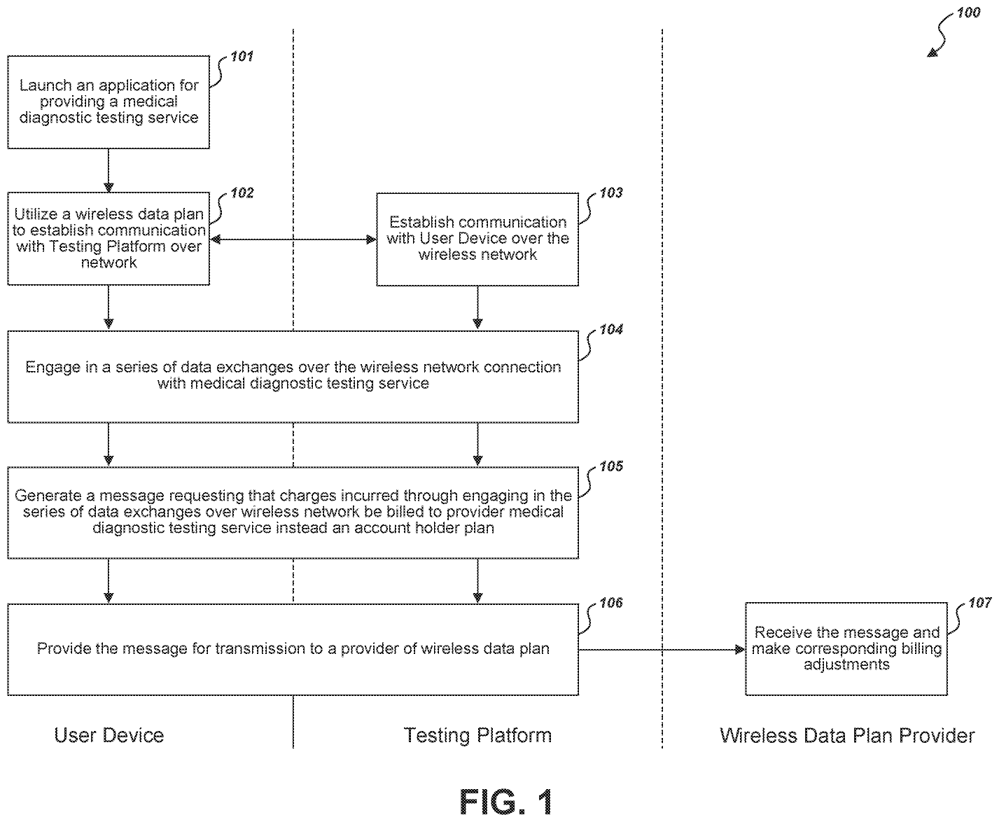

FIG. 1 provides a schematic diagram illustrating an embodiment of a testing and billing adjustment request process 100. As shown in FIG. 1, at block 101, a user may launch an application for providing a medical diagnostic testing service. The application can be an application that is stored and runs on a user device, such as a mobile phone, smartphone, tablet, laptop, personal computer, or other network connected devices. In some embodiments, the application can be web-based. The application may allow the user to engage in a testing session, which can, in some embodiments, be a proctored testing session. In some embodiments, a proctored testing session connects the user with a live or automated proctor that guides the user through one or more portions of a test. In some embodiments, a testing session need not include a proctor. For example, a user can be presented with textual, audio, or video instructions for taking a test without requiring a live proctor. In some embodiments, a live or automated proctor may review data that was generated or captured by the user device and/or the testing platform throughout the testing session.

At block 102, the user device may utilize a wireless data plan to establish communication with the testing platform over a network. The network can be, for example, a wireless cellular network, such as a 3G, 4G, 5G, LTE, or Edge network (among others), an internet based network, whether wired or wireless, or other types of networks. The network can allow the application (initiated at block 101) to communicate with one or more remotely located aspects of the testing platform. In response, the testing platform may at block 103 establish communication with the user device over the network.

As illustrated at block 104, the user device and the testing platform may then exchange data over the network in connection with the medical diagnostic testing service. Such data can, for example, facilitate a testing session. As illustrated at block 105, the user device and/or the testing platform may then generate a message requesting that charges incurred through engaging in the series of data exchanges over the network be billed to a provider of the medical diagnostic testing service instead of an account holder of the wireless data plan (such as the user). In some embodiments, the message generated at block 105 may relate to a single testing session or more than one testing session. As illustrated at block 106, the user device and/or the testing platform may then provide the message to a wireless data plan provider. As illustrated at block 107, the wireless data plan provider may receive the message and make corresponding billing adjustments. In some embodiments, the adjustments can include, for example, charging the testing platform for fees associated with data usage required for the test, thereby shifting the costs of using the testing platform from the user to the testing platform.

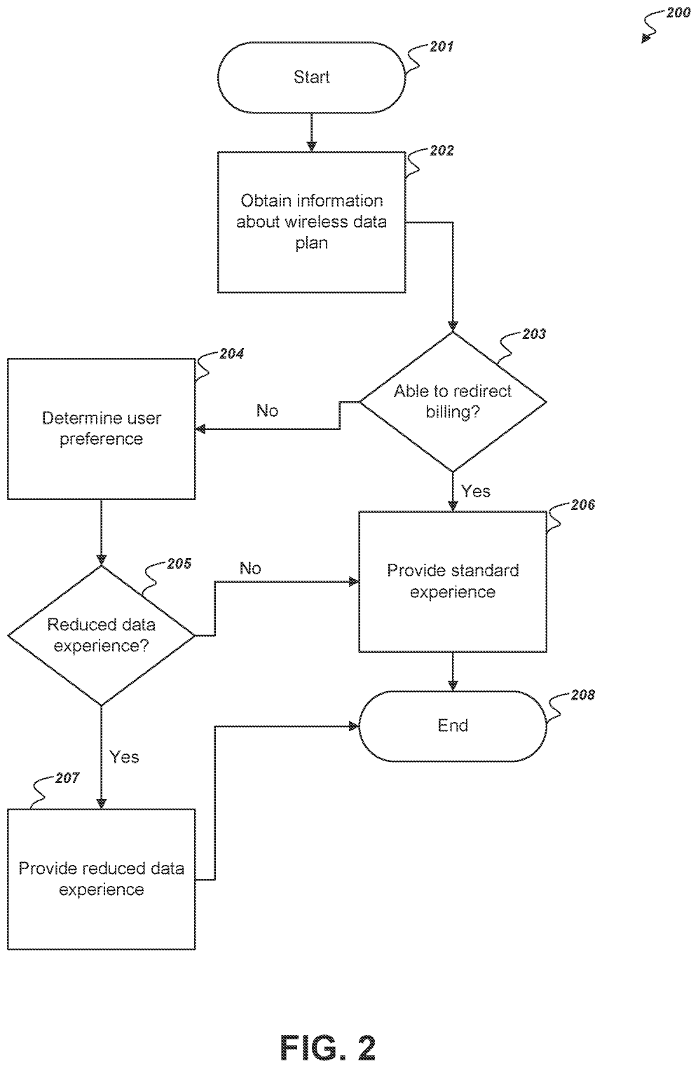

FIG. 2 shows a flowchart that depicts an example process 200 for selecting a test experience according to an embodiment which can be implemented on a computing system. The process can begin at block 201. In some embodiments, selection of a test experience can be based, for example, on a user's access to a data plan. At block 202, information may be obtained by the system relating to a user's wireless data plan. In some embodiments, the user can provide information manually. In some embodiments, information about the wireless data plan can be determined automatically. In some embodiments, the information can include, for example, the user's phone number, account number, or other information that may be used to identify the user or the account, the name of the wireless data plan provider, a data cap, and/or a data charge or the like. At block 203, the system may determine, based on the information about the wireless data plan, whether the billing may be redirected to a medical diagnostic testing service. If the billing can be redirected, the user may be provided with a standard or default testing experience at block 206. In some embodiments, if the billing cannot be redirected to a medical diagnostic testing service, the user may be asked at block 204 if they prefer a standard testing experience or a reduced data experience. In some embodiments, in response to the user's preference, the user may be presented with a standard testing experience or with a reduced data experience. In some embodiments, the standard experience may be different from the reduced data experience. In some embodiments, the standard experience may be the same as the reduced data experience such as, for example, if the standard experience for a particular test or tests does not use video, or if the standard experience is otherwise configured to use less data based on other factors such as, for example, available network bandwidth, network connection stability, lighting conditions, or the like.

At block 205, if the user does not prefer a reduced data experience, the system can provide a standard experience at block 206. If the user does prefer a reduced data experience, the system can provide a reduced data experience at block 207. After the user completes either the standard experience at block 206 or the reduced data experience at block 207, the process can end at block 208.

### Data Transfer Protocols

As mentioned briefly above, some embodiments relate to scenarios in which users of remote testing have limited cellular coverage or wireless internet access. Various embodiments described herein relate to systems, methods, and devices for collecting and transferring data in areas with insufficient cellular coverage or wireless internet access.

In some embodiments, a user device without a network connection (e.g., a device on which a user or users perform one or more tests) may periodically or continuously check whether access to a network connection is available. Once a network connection becomes available, the user device may transmit cached data over the network, for example, to a server of a testing platform. The server may receive the transmission of cached data. The cached data can then be deleted from the cache on the user device after the data has been successfully transmitted from the device.

For example, in some instances, a user may be guided through the administration of a medical exam, medical diagnostic test, or the like, with the use of a personal or user device (such as a cellphone, smartphone, tablet, laptop, personal digital assistant (PDA), or the like). The user device can be configured to gather information from or about the user during the testing session. Such information can include user input, pictures, video, geographical information, biometric data, among other types of information. The device can be configured to cache or save the data locally on the device. The user device can further be configured to transmit such information to, for example, a testing platform, over a network, such as a cellular network or internet-based network (e.g., 3G, 4G, 5G, LTE, EDGE, Wi-Fi, etc.) when a connection to such a network becomes available. In some instances, the user device can be configured to monitor or check, whether periodically or continuously, for a suitable network connection. Once a suitable connection becomes available, cached data can be uploaded over the network to a testing platform, and optionally, removed from the cache.

Users in developing regions and remote villages around the globe face certain technical constraints that hinder their ability to receive certain medical care and the user experience that is derived from the medical care provided. Some medical exams, medical diagnostic tests, or the like, are less intuitive than others and require a higher degree of assistance in the administration. A lack of sufficient cellular coverage or wireless internet access restricts users from interacting with various online platforms. Accordingly, it may be beneficial to administer guidance offline to enable to user to perform a medical exam, diagnostic test, or the like, and collect data locally until a network connection becomes available for transmission of the data collection. In this way, users in developing countries or regions and remote villages (or other areas where a network connection is not available) can be enabled to more easily interface with various online platforms, such as a remote testing platform.

In some embodiments, the systems, methods, and devices disclosed herein can be configured to enable the user to select which medical exam, medical diagnostic test, or the like, the user will be administering while not connected to a network. In some embodiments the user may be guided through how to perform the medical exam, medical diagnostic test, or the like, to ensure proper administration. In some embodiments, the selection and guidance of the medical exam, diagnostic test, or the like, can occur while the user is not connected to a network. During administration of the exam or test, data can be collected by the user device.

In some embodiments, the systems, methods, and devices disclosed herein include a cache or other memory or storage in the system. In some embodiments, the cache can be configured to store medical diagnostic testing data that is generated during administration of the testing session. For example, such data can include but is not limited to, test proctoring data, medical test data, imaging data of a test device (pictures, video, and/or audio), or the like. In some embodiments, the system can be configured to cache testing data in the cache until a network connection is available for transmitting the data to a server system. In some embodiments, the systems disclosed herein can be configured to determine periodically or in substantially real-time or to dynamically assess whether there is a stable electronic network connection for transferring the testing data to a server system, such as a testing platform. In some embodiments, the systems disclosed herein can be configured to determine whether there is a stable electronic network connection and transfer the testing data stored in the cache to a central server.

In some embodiments, data from multiple medical diagnostic testing sessions can be collected and cached locally prior to establishing a network connection. In some embodiments, the medical diagnostic testing session can be administered offline. In some embodiments, the cache can be configured to store medical diagnostic testing data from multiple users prior to establishing a stable connection that allows for transferring the testing data of multiple users to the server system. In some embodiments, the systems, methods, and devices described herein can prioritize data within or across each medical diagnostic testing session. When a network connection is established, depending on the strength of the network connection, high priority data within each medical diagnostic testing session can be transferred to the server before lower priority data is transferred. In this embodiment, if the electronic network connection is unstable or weak, the high priority data cached locally can be transferred to the server, while the lower priority data cached locally can remain stored.

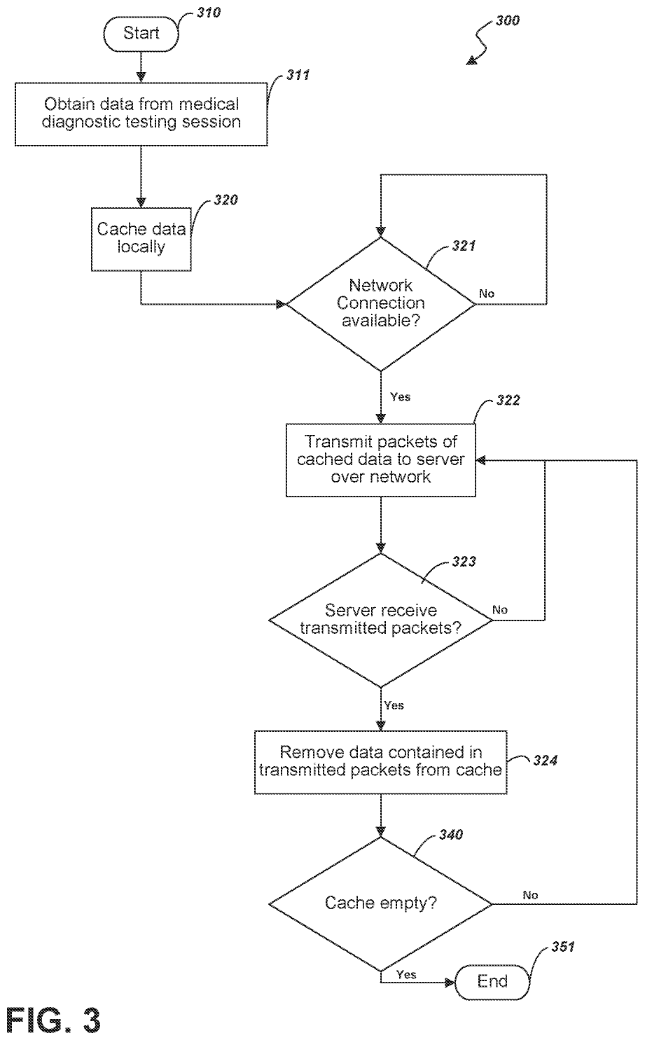

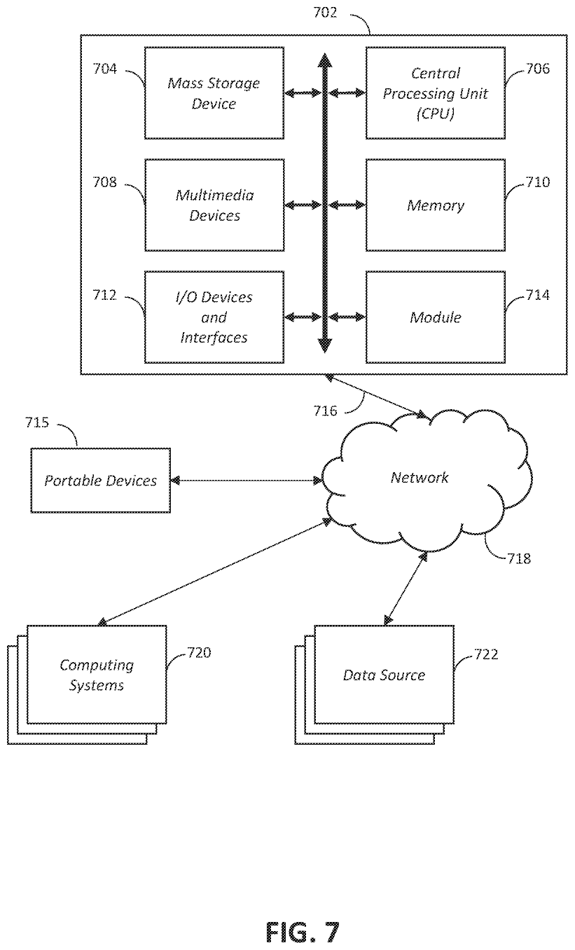

FIG. 3 is a flowchart illustrating an example data transfer protocol or method 300. The method 300 can be configured to facilitate administration of remote testing sessions, even when a reliable or suitable network connection is not available. The method 300 can be implemented on a user device (e.g., user device 715 of FIG. 7). The user device can be a cell phone, laptop, desktop, tablet, and/or the like. The user device can be configured to guide a user through a remote testing session, such as a health exam or diagnostic. The user device can provide such guidance even when no network connection is available.

After beginning at block 310, at block 311, the user device obtains data from a medical diagnostic testing session. The data can include user inputs entered via the user device or other data gathered by the user device, such as audio, video, pictures, biometric data, health measurements, etc. At block 320, the data can be cached locally on the user device. For example, the data can be saved to a cache or memory of the user device.

At block 321, the user device monitors for whether a network connection is available. Monitoring can involve determining one or more of the presence of a network, a type of the network, a speed of the network, the stability of the network, the bandwidth of the network, or the like. The user device can assess the network to determine whether it is sufficient for uploading cached data.

At block 322, if a suitable network is available, the user device transmits the cached data over the network. In some embodiments, transmission can be accomplished by sending one or more packets of data over the network. At block 323, the user device can check (e.g., monitor for a confirmation) whether the transmitted packets (block 322) have been received by a remote server. If the packets are not received by the remote server, they can be resent by the user device. At block 324, if the packets are received by the remote server, they can then be removed from the cache of the user device. This can free up additional space on the user device for additional testing. At block 340, the process can continue until the entire cache is empty and the process ends at block 351.

The method 300 can advantageously provide for transmission of data over a network, even if the network is somewhat unstable. The method 300 will continue to transmit data, while network connections are available, until it receives confirmation that the data has been received by the remote server. At which time, the user device can clear the data from its cache. In this way, in some embodiments, all data in the cache still needs to be sent, and, when the cache is empty, all data has been successfully transferred to the remote server.

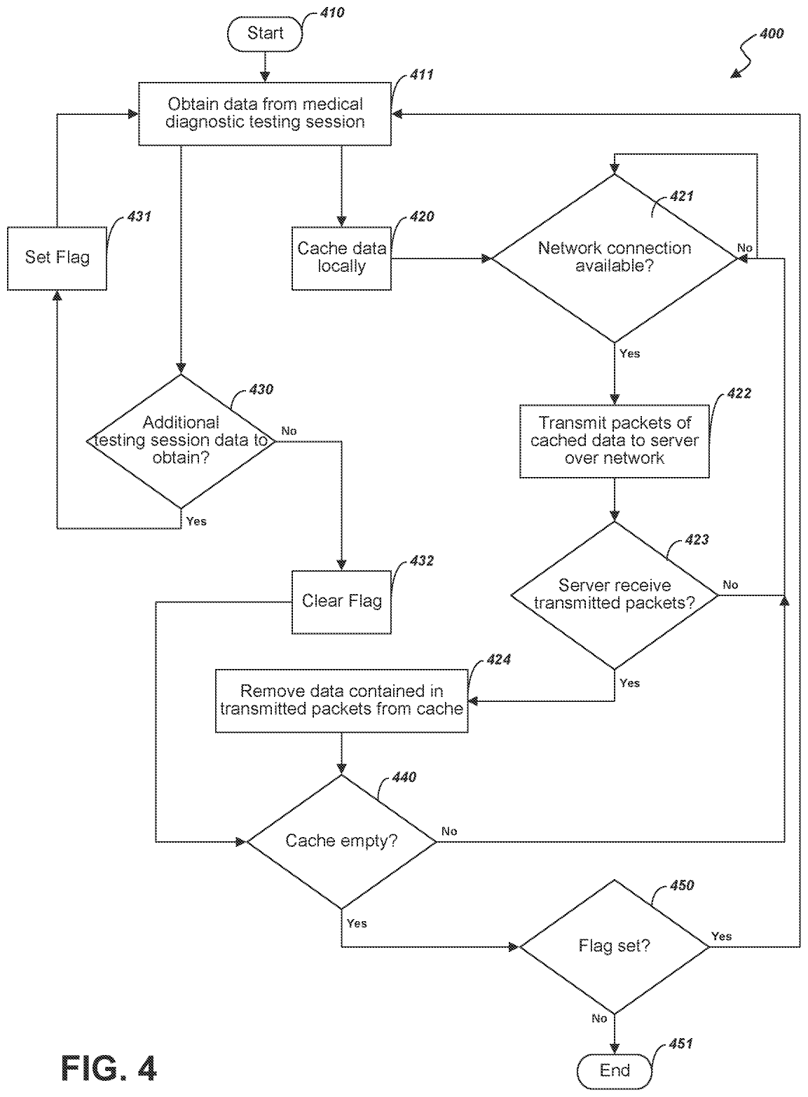

FIG. 4 is a flowchart for another embodiment of a data transfer protocol or method 400. The method can begin at block 410. In this example, the method 400 is configured such that a user device can be used for multiple testing sessions. The multiple testing sessions can accommodate a plurality of users. The data from the multiple testing sessions can be gathered by a user device and stored locally until a suitable network connection becomes available. When a suitable network connection becomes available, the data can be uploaded over the network and cleared from the user device cache.

In some embodiments, data is first obtained, by the user device, from a medical diagnostic testing session at block 411. Data can be obtained on a user device that is configured to provide the testing session as described above. This can be data from a first testing session. Once data from a medical diagnostic testing session is obtained, the data can be cached locally, as illustrated at block 420. At block 421, the protocol can determine whether a network connection is available (e.g., by performing one or more operations similar or equivalent to those described above with reference to block 321 of FIG. 3). In some embodiments, a user may administer a medical exam, diagnostic test, or the like, while the method 400 is simultaneously assessing whether a network connection is available to transfer a previous user's medical diagnostic testing session data. If a network connection is available, the method 400 can include a transmission of packets of cached data to a server over a network, as illustrated at block 422. However, if there is no network connection available, as illustrated in block 421, the method 400 can continue to assess (whether continuously or periodically) whether a network connection is available until there is a network connection available.

As illustrated at block 422, cached data can be sent over the network in packets when a network connection is available. In some instances, the data that is transmitted at block 422 may only represent a portion of the data that is stored in the cache. At block 423, the user device can determine whether the server received the transmitted packets of cached data. For example, the user device may receive an acknowledgement from the server notifying the user device that it received the transmitted packets of cached data. If the server received the transmitted packets of cached data, the data contained in the transmitted packets of cached data may be removed from the cache at block 424. If the user device determines (e.g., by lack of response) that the server has not received the transmitted data, the method 400 can return to block 421 and continue to monitor for a network connection, resending the data packets at block 422 once a network connection is reestablished.

Returning to block 411, in some embodiments, the method 400 may also move from block 411 to block 430. At block 430, the user device determines whether additional testing session data needs to be obtained. This can be the case, for example, where the testing session is ongoing. In such a case, a flag is set at block 431 indicating that additional testing session data is still being obtained. This can continue until the testing session is completed. At which point, the flag at block 432 can be cleared, indicating that no further testing session data is to be obtained.

In some embodiments, the additional testing session data can be data from a second testing session, which can be performed by the original user or a second user. In this way, the user device can be used to provide testing for multiple users.

Returning to block 440, the method 400 may determine whether the cache is empty. If the method 400 determines that the cache is not empty at block 440, the method 400 may return to block 421 to determine whether a network connection is available. The method 400 may continue to attempt to empty the cache until the cache is empty as determined at block 440. If the cache is empty, the method 400 may check whether the flag is set at block 450. If a flag is not set, the method 400 may terminate at block 451. If a flag is set, then the method 400 may restart at block 411 to obtain data from a medical diagnostic testing session.

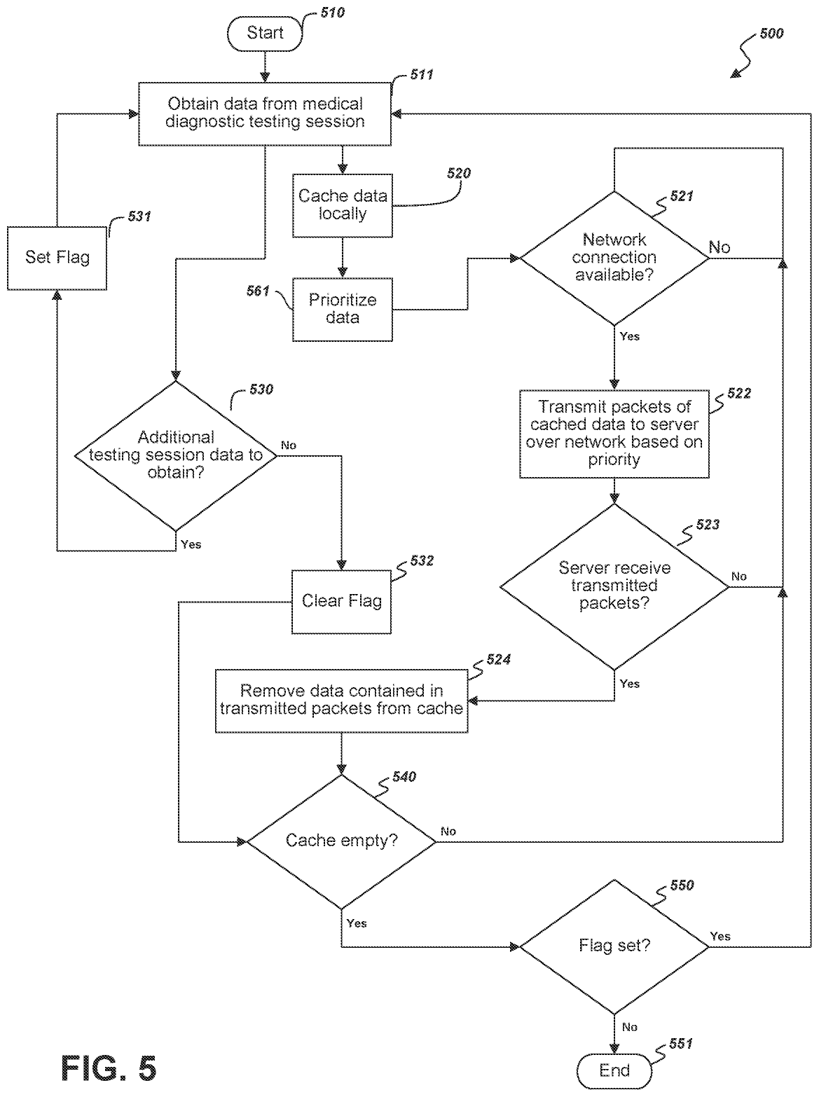

FIG. 5 is a flowchart for another embodiment of a data transfer protocol or method 500. In this example, the method 500 is configured to prioritize data for upload. In many respects, the method 500 is similar to the method 400. Similar aspects will not be described. As shown at block 561, as data is generated and stored during a testing session, such data can be prioritized. For example, certain data may be indicated to be higher priority and other data may be indicated to be lower priority. Prioritization of the data can be used to determine, at block 522, the order in which packets of data are transmitted over the network. For example, higher priority data can be sent before lower priority data. In this way, if a network connection is unstable, higher priority data can be sent first in case the network connection drops. Data prioritization can include data related to key aspects of the testing session, such as data from which test results can be determined. In some embodiments, data prioritization can include identifying one or more portions of a video or one or more images from a video. These can be video portions or images that can be reviewed to determine whether the test was administered correctly.

### Remotely-Deployable Testing Apparatus

As discussed briefly above, users of remote medical testing may be located in areas that have limited physical accessibility and/or internet connectivity, making delivery of test supplies and/or retrieval of test results or collected samples difficult. In some embodiments, a remotely-deployable testing apparatus may be delivered to a remote region using, for example, airdrops or drones. Test takers in remote areas may not have devices such a smartphones, tablets, laptops, desktops, or other computing devices that can be used to take remote medical tests. Thus, in some embodiments, remotely-deployable testing apparatuses may include computing devices. In some embodiments, internet access may be limited or unavailable, and remotely-deployable testing apparatuses may store results and other testing information (for example, video of testing sessions) until internet access is available, at which time the test results and other information may be uploaded to a server. In some embodiments, testing may be done entirely at the remote location.

In some embodiments, it may be advantageous to associate test takers with particular tests. For example, test takers may need their test results to obtain treatment or to access facilities. Moreover, some tests cannot be performed entirely remotely. Thus, in some embodiments, samples may be collected at the remote location and subsequently sent to a laboratory facility for testing.

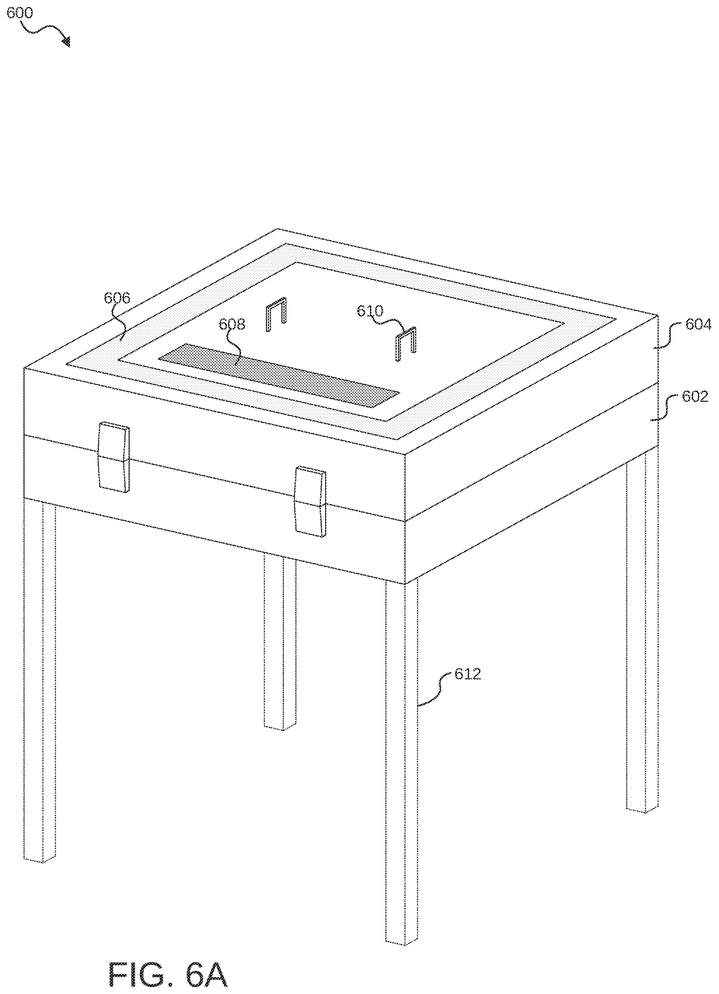

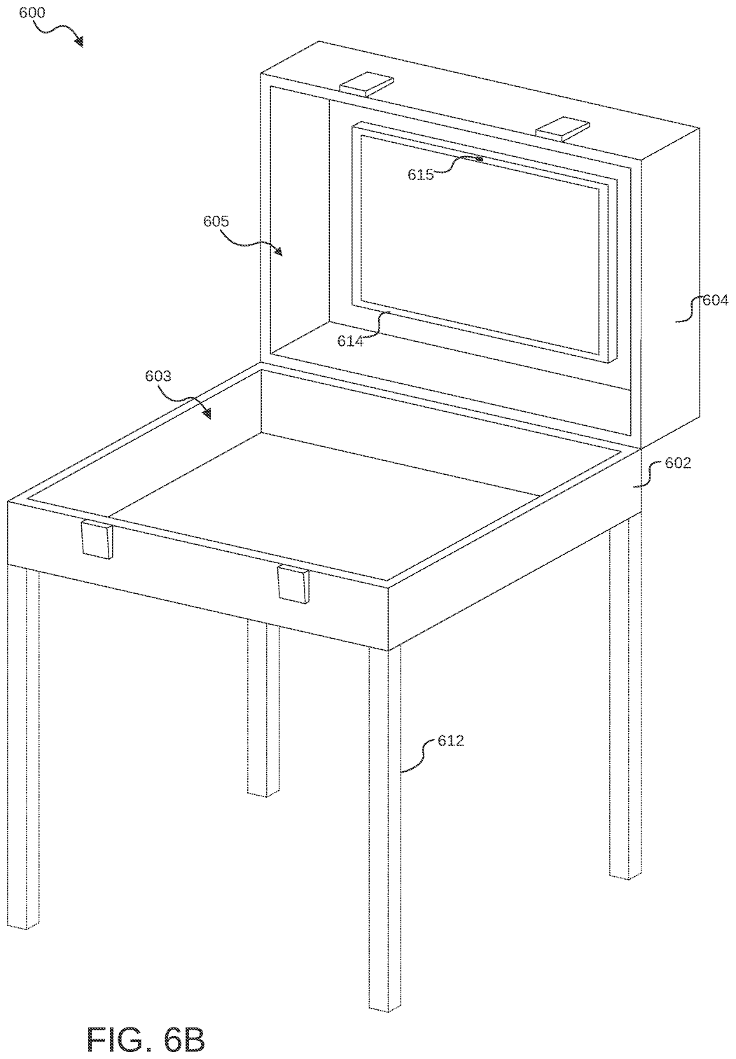

FIG. 6A is an example embodiment of a remotely-deployable testing apparatus in a closed state. As shown in FIG. 6A, a remotely-deployable testing apparatus 600 may comprise a container. The container may comprise a lower outer container portion 602, an upper outer container portion 604, one or more fiducial markers 606 on one or more exterior surfaces, one or more solar cells 608 and associated hardware, one or more hardware components 610 (for example, hooks) for coupling to a drone or other delivery device, and two or more legs 612 that are mounted to the underside of the lower outer container portion 602.

In some embodiments, the hardware components 610 may be integrated into a handle component for carrying the apparatus 600. In some embodiments, the system may be configured for delivery and/or pickup by, for example, airdrop, drone, truck, foot, or the like. The system may be equipped with hardware components for coupling with a drone, a parachute, or the like. In some embodiments, fiducial markers may be used to aid in locating and picking up the system such as by, for example, a drone. In some embodiments, the legs 612 may be detachable, collapsible, and/or foldable. In some embodiments, the legs 612 may enable the apparatus 600 to serve as a kiosk that users may interact with.

FIG. 6B is an example embodiment of a remotely-deployable testing apparatus in an open state. The apparatus 600 may comprise a lower inner container portion 603 (e.g., a cavity) which may contain, for example, testing supplies, and an upper inner container portion 605 containing a computing device 614 and a camera 615. The computing device 614 may be, for example, a smartphone, tablet, laptop, or other computing device. The camera 615 may be integrated into the computing device 614 or may be external to the computing device 614 and connected to the computing device 614 using, for example, a USB connection. In some embodiments, the testing apparatus may include a tray or surface that may fold or slide out and be used as a testing surface.

In some embodiments, the container of the system may be ruggedized to withstand drops, water, dust, extreme heat, extreme cold, or the like in order to protect the contents from damage. In some embodiments, the system may be equipped with one or more sensors such as, for example, drop indicator tags and/or time/temperature indicator tags. These tags may indicate that a container has been dropped or that the container has exceeded a maximum and/or minimum temperature for a period of time. In some embodiments, indicators may be used to determine if test materials or collected samples are usable. For example, for some types of diagnostic tests, samples must be kept below a certain temperature to prevent degradation of the samples. Similarly, test materials may need to be maintained in a limited temperature range.

In some embodiments, the system may include health-related materials such as diagnostic tests (for example, tests for malaria, COVID-19, or other conditions). In some embodiments, the system may contain personal protective equipment such as gloves, masks, and the like. In some embodiments, the system may include basic first aid items such as bandages and/or tools for filtering and purifying water.

In some embodiments, the system may contain one or more computing devices and/or communication devices. For example, in some embodiments, the system may include a tablet, smartphone, or other computing device that can be used to take diagnostic tests. In some embodiments, if a network connection is available, the system may be configured to allow test takers to connect with remote doctors, nurses, proctors, or other medical staff. In some embodiments, the computing device may be configured to enable users to take diagnostic tests without an internet connection. The test results may be stored locally and then automatically uploaded once an internet connection can be established, for example using one or more of the data transfer protocols described above. In some embodiments, the computing device may be stripped of some or all non-essential features in order to deter theft. For example, the computing device may be restricted to running only the software needed for taking tests, and/or one or more hardware components may be disabled or removed, such as rear-facing cameras, Bluetooth modules, or other components that are not needed for testing.

In some embodiments, the system may contain equipment such as a satellite phone, GPS, or other components configured to communicate with one or more satellites. In some embodiments, the system may include hardware for deploying a wireless local area network (WLAN). The WLAN may be used to enable personal devices belonging to test takers to be used for testing purposes. For example, test takers may use their personal devices to take tests and may transfer test information (for example, video of the testing session) over the WLAN. In some embodiments, the system may comprise additional components such as a pre-charged battery that can be used to charge a phone, tablet, or other electronic device. In some embodiments, the system may include one or more solar cells (also referred to herein as solar panels) and associated components to power other electronic devices or components. In some embodiments, the system can include a charge controller that can be used to regulate a voltage, current, power, and so forth of the solar cells.

In some embodiments, it may be preferable to associate one or more test results with one or more test takers. For example, for some types of tests, sample collection can be done remotely, but samples must be sent to a laboratory for testing. In some circumstances, test takers may need to a record of their test results in the future. For example, proof of a negative test may be required to gain entrance to a facility, or proof of a positive test may be required to obtain treatment. Accordingly, in some embodiments, the system may contain one or more items that can be used for tracking and associating tests with individuals. For example, the system may contain bracelets, cards, fobs, masks, or other artifacts that may be used to associate individuals with tests. For example, the artifacts may contain passive radio frequency identification tags (RFID tags) or be marked with QR codes or other codes. Alternatively or additionally, photo identification or other documents, such as a government-issued ID card, may be used. For example, a test taker may present proof of identification when taking a test, which may be stored with the test result and/or associated with an account of the test taker. In some embodiments, biometrics may be collected as part of the testing process. For example, the test taker may be asked to capture a photo using the camera of the computing device and/or to capture one or more fingerprints using a fingerprint reader. Biometric data may be used to verify the user when results are retrieved and/or may be used as login credentials to access test results. In some embodiments, geolocation and/or image data may be used. For example, if a user takes a test at his or her residence, geolocation information may be recorded along with one or more images of the residence. This information may be used to locate the test taker in order to, for example, deliver test results or treatments. Combinations of the above embodiments may be advantageous. For example, geolocation data may be used alongside RFID or QR codes to locate individual test takers more easily.

In some embodiments, rather than, or in addition to, accessing test results stored on a remote system, it may be advantageous to provide users with artifacts that include test results. For example, in some embodiments, an RFID writer can be used to encode one-time writable RFID tags with the test taker's personal information and test results, eliminating the need to contact a server to obtain test results. Similarly, in some embodiments, the test taker's personal information and/or test results may be used to generate a printable code such as a QR code, barcode, data matrix, or other code that can be printed by a printer that may be included in the remotely-deployable testing apparatus.

In some embodiments, the system may include one or more features designed to improve safety. For example, in some embodiments, the system may include sterilization equipment such as an ultraviolet (UV) lighting system. For example, the system may include a dedicated compartment to use for UV sterilization. In some embodiments, the system may include a receptacle to allow for the safe disposal of used testing materials and other medical waste. In some embodiments, individual swabs, syringes, and the like may have unique RFID tags, QR codes, and/or other identifiers that may be used to ensure that testing supplies are not reused.

In some embodiments, the system may include the ability interface with, for example, a drone-based prescription and/or treatment delivery system.

### Computer Systems

FIG. 7 is a block diagram depicting an embodiment of a computer hardware system configured to run software for implementing one or more embodiments disclosed herein.

In some embodiments, the systems, processes, and methods described herein are implemented using a computing system, such as the one illustrated in FIG. 7. The example computer system 702 is in communication with one or more computing systems 720 and/or one or more data sources 722 via one or more networks 718. While FIG. 7 illustrates an embodiment of a computing system 702, it is recognized that the functionality provided for in the components and modules of computer system 702 may be combined into fewer components and modules, or further separated into additional components and modules.

The computer system 702 can comprise a module 714 that carries out the functions, methods, acts, and/or processes described herein. The module 714 is executed on the computer system 702 by a central processing unit 706 discussed further below.

In general, the word “module,” as used herein, refers to logic embodied in hardware or firmware or to a collection of software instructions, having entry and exit points. Modules are written in a program language, such as JAVA, C or C++, Python, or the like. Software modules may be compiled or linked into an executable program, installed in a dynamic link library, or may be written in an interpreted language such as BASIC, PERL, LUA, or Python. Software modules may be called from other modules or from themselves, and/or may be invoked in response to detected events or interruptions. Modules implemented in hardware include connected logic units such as gates and flip-flops, and/or may include programmable units, such as programmable gate arrays or processors.

Generally, the modules described herein refer to logical modules that may be combined with other modules or divided into sub-modules despite their physical organization or storage. The modules are executed by one or more computing systems and may be stored on or within any suitable computer readable medium or implemented in-whole or in-part within special designed hardware or firmware. Not all calculations, analysis, and/or optimization require the use of computer systems, though any of the above-described methods, calculations, processes, or analyses may be facilitated through the use of computers. Further, in some embodiments, process blocks described herein may be altered, rearranged, combined, and/or omitted.

The computer system 702 includes one or more processing units (CPU) 706, which may comprise a microprocessor. The computer system 702 further includes a physical memory 710, such as random-access memory (RAM) for temporary storage of information, a read only memory (ROM) for permanent storage of information, and a mass storage device 704, such as a backing store, hard drive, rotating magnetic disks, solid state disks (SSD), flash memory, phase-change memory (PCM), 3D XPoint memory, diskette, or optical media storage device. Alternatively, the mass storage device may be implemented in an array of servers. Typically, the components of the computer system 702 are connected to the computer using a standards-based bus system. The bus system can be implemented using various protocols, such as Peripheral Component Interconnect (PCI), Micro Channel, SCSI, Industrial Standard Architecture (ISA) and Extended ISA (EISA) architectures.

The computer system 702 includes one or more input/output (I/O) devices and interfaces 712, such as a keyboard, mouse, touch pad, and printer. The I/O devices and interfaces 712 can include one or more display devices, such as a monitor, that allows the visual presentation of data to a user. More particularly, a display device provides for the presentation of GUIs as application software data, and multi-media presentations, for example. The I/O devices and interfaces 712 can also provide a communications interface to various external devices. The computer system 702 may comprise one or more multi-media devices 708, such as speakers, video cards, graphics accelerators, and microphones, for example.

The computer system 702 may run on a variety of computing devices, such as a server, a Windows server, a Structure Query Language server, a Unix Server, a personal computer, a laptop computer, and so forth. In other embodiments, the computer system 702 may run on a cluster computer system, a mainframe computer system and/or other computing system suitable for controlling and/or communicating with large databases, performing high volume transaction processing, and generating reports from large databases. The computing system 702 is generally controlled and coordinated by an operating system software, such as Windows XP, Windows Vista, Windows 7, Windows 8, Windows 10, Windows 11, Windows Server, Unix, Linux (and its variants such as Debian, Linux Mint, Fedora, and Red Hat), SunOS, Solaris, Blackberry OS, z/OS, i0S, macOS, or other operating systems, including proprietary operating systems. Operating systems control and schedule computer processes for execution, perform memory management, provide file system, networking, and I/O services, and provide a user interface, such as a graphical user interface (GUI), among other things.

The computer system 702 illustrated in FIG. 7 is coupled to a network 718, such as a LAN, WAN, or the Internet via a communication link 716 (wired, wireless, or a combination thereof). Network 718 communicates with various computing devices and/or other electronic devices. Network 718 is communicating with one or more computing systems 720 and one or more data sources 722. The module 714 may access or may be accessed by computing systems 720 and/or data sources 722 through a web-enabled user access point. Connections may be a direct physical connection, a virtual connection, and other connection type. The web-enabled user access point may comprise a browser module that uses text, graphics, audio, video, and other media to present data and to allow interaction with data via the network 718.

Access to the module 714 of the computer system 702 by computing systems 720 and/or by data sources 722 may be through a web-enabled user access point such as the computing systems' 720 or data source's 722 personal computer, cellular phone, smartphone, laptop, tablet computer, e-reader device, audio player, or another device capable of connecting to the network 718. Such a device may have a browser module that is implemented as a module that uses text, graphics, audio, video, and other media to present data and to allow interaction with data via the network 718.

The output module may be implemented as a combination of an all-points addressable display such as a cathode ray tube (CRT), a liquid crystal display (LCD), a plasma display, or other types and/or combinations of displays. The output module may be implemented to communicate with input devices 712 and they also include software with the appropriate interfaces which allow a user to access data through the use of stylized screen elements, such as menus, windows, dialogue boxes, tool bars, and controls (for example, radio buttons, check boxes, sliding scales, and so forth). Furthermore, the output module may communicate with a set of input and output devices to receive signals from the user.

The input device(s) may comprise a keyboard, roller ball, pen and stylus, mouse, trackball, voice recognition system, or pre-designated switches or buttons. The output device(s) may comprise a speaker, a display screen, a printer, or a voice synthesizer. In addition, a touch screen may act as a hybrid input/output device. In another embodiment, a user may interact with the system more directly such as through a system terminal connected to the score generator without communications over the Internet, a WAN, or LAN, or similar network.

In some embodiments, the system 702 may comprise a physical or logical connection established between a remote microprocessor and a mainframe host computer for the express purpose of uploading, downloading, or viewing interactive data and databases on-line in real time. The remote microprocessor may be operated by an entity operating the computer system 702, including the client server systems or the main server system, an/or may be operated by one or more of the data sources 722 and/or one or more of the computing systems 720. In some embodiments, terminal emulation software may be used on the microprocessor for participating in the micro-mainframe link.

In some embodiments, computing systems 720 who are internal to an entity operating the computer system 702 may access the module 714 internally as an application or process run by the CPU 706.

In some embodiments, one or more features of the systems, methods, and devices described herein can utilize a URL and/or cookies, for example for storing and/or transmitting data or user information. A Uniform Resource Locator (URL) can include a web address and/or a reference to a web resource that is stored on a database and/or a server. The URL can specify the location of the resource on a computer and/or a computer network. The URL can include a mechanism to retrieve the network resource. The source of the network resource can receive a URL, identify the location of the web resource, and transmit the web resource back to the requestor. A URL can be converted to an IP address, and a Domain Name System (DNS) can look up the URL and its corresponding IP address. URLs can be references to web pages, file transfers, emails, database accesses, and other applications. The URLs can include a sequence of characters that identify a path, domain name, a file extension, a host name, a query, a fragment, scheme, a protocol identifier, a port number, a username, a password, a flag, an object, a resource name and/or the like. The systems disclosed herein can generate, receive, transmit, apply, parse, serialize, render, and/or perform an action on a URL.

A cookie, also referred to as an HTTP cookie, a web cookie, an internet cookie, and a browser cookie, can include data sent from a website and/or stored on a user's computer. This data can be stored by a user's web browser while the user is browsing. The cookies can include useful information for websites to remember prior browsing information, such as a shopping cart on an online store, clicking of buttons, login information, and/or records of web pages or network resources visited in the past. Cookies can also include information that the user enters, such as names, addresses, passwords, credit card information, etc. Cookies can also perform computer functions. For example, authentication cookies can be used by applications (for example, a web browser) to identify whether the user is already logged in (for example, to a web site). The cookie data can be encrypted to provide security for the consumer. Tracking cookies can be used to compile historical browsing histories of individuals. Systems disclosed herein can generate and use cookies to access data of an individual. Systems can also generate and use JSON web tokens to store authenticity information, HTTP authentication as authentication protocols, IP addresses to track session or identity information, URLs, and the like.

The computing system 702 may include one or more internal and/or external data sources (for example, data sources 722). In some embodiments, one or more of the data repositories and the data sources described above may be implemented using a relational database, such as Sybase, Oracle, CodeBase, DB2, PostgreSQL, and Microsoft® SQL Server as well as other types of databases such as, for example, a NoSQL database (for example, Couchbase, Cassandra, or MongoDB), a flat file database, an entity-relationship database, an object-oriented database (for example, InterSystems Cache), a cloud-based database (for example, Amazon RDS, Azure SQL, Microsoft Cosmos DB, Azure Database for MySQL, Azure Database for MariaDB, Azure Cache for Redis, Azure Managed Instance for Apache Cassandra, Google Bare Metal Solution for Oracle on Google Cloud, Google Cloud SQL, Google Cloud Spanner, Google Cloud Big Table, Google Firestore, Google Firebase Realtime Database, Google Memorystore, Google MongoDB Atlas, Amazon Aurora, Amazon DynamoDB, Amazon Redshift, Amazon ElastiCache, Amazon MemoryDB for Redis, Amazon DocumentDB, Amazon Keyspaces, Amazon Neptune, Amazon Timestream, or Amazon QLDB), a non-relational database, or a record-based database.

The computer system 702 may also access one or more databases 722. The databases 722 may be stored in a database or data repository. The computer system 702 may access the one or more databases 722 through a network 718 or may directly access the database or data repository through I/O devices and interfaces 712. The data repository storing the one or more databases 722 may reside within the computer system 702.

### Additional Embodiments

In the foregoing specification, the systems and processes have been described with reference to specific embodiments thereof. It will, however, be evident that various modifications and changes may be made thereto without departing from the broader spirit and scope of the embodiments disclosed herein. The specification and drawings are, accordingly, to be regarded in an illustrative rather than restrictive sense.

Indeed, although the systems and processes have been disclosed in the context of certain embodiments and examples, it will be understood by those skilled in the art that the various embodiments of the systems and processes extend beyond the specifically disclosed embodiments to other alternative embodiments and/or uses of the systems and processes and obvious modifications and equivalents thereof. In addition, while several variations of the embodiments of the systems and processes have been shown and described in detail, other modifications, which are within the scope of this disclosure, will be readily apparent to those of skill in the art based upon this disclosure. It is also contemplated that various combinations or sub-combinations of the specific features and aspects of the embodiments may be made and still fall within the scope of the disclosure. It should be understood that various features and aspects of the disclosed embodiments can be combined with, or substituted for, one another in order to form varying modes of the embodiments of the disclosed systems and processes. Any methods disclosed herein need not be performed in the order recited. Thus, it is intended that the scope of the systems and processes herein disclosed should not be limited by the particular embodiments described above.

It will be appreciated that the systems and methods of the disclosure each have several innovative aspects, no single one of which is solely responsible or required for the desirable attributes disclosed herein. The various features and processes described above may be used independently of one another or may be combined in various ways. All possible combinations and sub-combinations are intended to fall within the scope of this disclosure.

Certain features that are described in this specification in the context of separate embodiments also may be implemented in combination in a single embodiment. Conversely, various features that are described in the context of a single embodiment also may be implemented in multiple embodiments separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination. No single feature or group of features is necessary or indispensable to each and every embodiment.

It will also be appreciated that conditional language used herein, such as, among others, “can,” “could,” “might,” “may,” “for example,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open- ended fashion, and do not exclude additional elements, features, acts, operations, and so forth. In addition, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise. Similarly, while operations may be depicted in the drawings in a particular order, it is to be recognized that such operations need not be performed in the particular order shown or in sequential order, or that all illustrated operations be performed, to achieve desirable results. Further, the drawings may schematically depict one or more example processes in the form of a flowchart. However, other operations that are not depicted may be incorporated in the example methods and processes that are schematically illustrated. For example, one or more additional operations may be performed before, after, simultaneously, or between any of the illustrated operations. Additionally, the operations may be rearranged or reordered in other embodiments. In certain circumstances, multitasking and parallel processing may be advantageous. Moreover, the separation of various system components in the embodiments described above should not be understood as requiring such separation in all embodiments, and it should be understood that the described program components and systems may generally be integrated together in a single software product or packaged into multiple software products. Additionally, other embodiments are within the scope of the following claims. In some cases, the actions recited in the claims may be performed in a different order and still achieve desirable results.

Further, while the methods and devices described herein may be susceptible to various modifications and alternative forms, specific examples thereof have been shown in the drawings and are herein described in detail. It should be understood, however, that the embodiments are not to be limited to the particular forms or methods disclosed, but, to the contrary, the embodiments are to cover all modifications, equivalents, and alternatives falling within the spirit and scope of the various implementations described and the appended claims. Further, the disclosure herein of any particular feature, aspect, method, property, characteristic, quality, attribute, element, or the like in connection with an implementation or embodiment can be used in all other implementations or embodiments set forth herein. Any methods disclosed herein need not be performed in the order recited. The methods disclosed herein may include certain actions taken by a practitioner; however, the methods can also include any third-party instruction of those actions, either expressly or by implication. The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (for example, as accurate as reasonably possible under the circumstances, for example ±5%, ±10%, ±15%, etc.). For example, “about 3.5 mm” includes “3.5 mm.” Phrases preceded by a term such as “substantially” include the recited phrase and should be interpreted based on the circumstances (for example, as much as reasonably possible under the circumstances). For example, “substantially constant” includes “constant.” Unless stated otherwise, all measurements are at standard conditions including temperature and pressure.

As used herein, a phrase referring to “at least one of” a list of items refers to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain embodiments require at least one of X, at least one of Y, and at least one of Z to each be present. The headings provided herein, if any, are for convenience only and do not necessarily affect the scope or meaning of the devices and methods disclosed herein.

Accordingly, the claims are not intended to be limited to the embodiments shown herein but are to be accorded the widest scope consistent with this disclosure, the principles and the novel features disclosed herein.

### Example Clauses:

Examples of the implementations of the present disclosure can be described in view of the following example clauses. The features recited in the below example implementations can be combined with additional features disclosed herein. Furthermore, additional inventive combinations of features are disclosed herein, which are not specifically recited in the below example implementations, and which do not include the same features as the specific implementations below. For sake of brevity, the below example implementations do not identify every inventive aspect of this disclosure. The below example implementations are not intended to identify key features or essential features of any subject matter described herein. Any of the example clauses below, or any features of the example clauses, can be combined with any one or more other example clauses, or features of the example clauses or other features of the present disclosure.

Clause 1. A method for redirecting billing by a wireless data plan provider to a medical diagnostic testing service, the method comprising: exchanging data between a testing platform and a user device; generating a message requesting that charges associated with a remote medical diagnostic test be billed to the medical diagnostic testing service; and transmitting the message from the testing platform to the wireless data plan provider.

Clause 2. The method of clause 1, wherein the message is generated by the testing platform.

Clause 3. The method of clause 1, wherein the message is generated by the user device.

Clause 4. The method of clause 1, wherein the message is transmitted to the wireless data plan provider by the testing platform.

Clause 5. The method of clause 1, wherein the message is transmitted to the wireless data plan provider by the user device.

Clause 6. The method of clause 1, wherein the message comprises an amount of data used by the remote medical diagnostic test.

Clause 7. The method of clause 1, wherein the message comprises an estimated amount of data used by the remote medical diagnostic test.

Clause 8. The method of clause 1, wherein the message comprises a start time of the remote medical diagnostic test and an end time of the remote medical diagnostic test.

Clause 9. The method of clause 1, wherein the message comprises an amount of data used by a plurality of remote medical diagnostic tests.

Clause 10. The method of clause 1, wherein the message comprises a maximum data usage amount.

Clause 11. The method of clause 1, wherein the message comprises an estimate of an amount of data used by a plurality of remote medical diagnostic tests.

Clause 12. The method of clause 1, wherein the message comprises a plurality of start times and a plurality of end times of a plurality of remote medical diagnostic tests.

Clause 13. A system for cost-shifting data used for a remote medical diagnostic test comprising: a non-transitory computer-readable medium with instructions encoded thereon; and one or more processors configured to execute the instructions to cause the system to: receive, from a user device, identifying information indicative of a wireless data plan associated with the user device; provide the remote medical diagnostic test to a user via a user device; receive, from the user device, data related to the medical diagnostic test; determine an indication of an amount of data used for the remote medical diagnostic test; generate a message to request cost-shifting of the amount of data from the user to a testing platform provider; and send the message to a provider of the wireless data plan.

Clause 14. The system of clause 13, wherein the message comprises the indication of the amount of data used for the remote medical diagnostic test.

Clause 15. The system of clause 13, wherein the message comprises identifying information indicative of the wireless data plan.

Clause 16. The system of clause 13, wherein the indication of the amount of data used for the remote medical diagnostic test comprises an amount of data used for the remote medical diagnostic test.

Clause 17. The system of clause 13, wherein the indication of the amount of data used for the remote medical diagnostic test comprises an estimated amount of data used for the remote medical diagnostic test.

Clause 18. The system of clause 13, wherein the indication of the amount of data used for the remote medical diagnostic test comprises a start time and a stop time of the remote medical diagnostic test.

Clause 19. The system of clause 13, wherein message comprises a maximum data usage amount.

Clause 20. The system of clause 13, wherein the non-transitory computer-readable medium has instructions that encoded thereon that, when executed by the one or more processors, cause the system to: determine, based on the identifying information, that cost-shifting is not available for the medical diagnostic test; and provide, to the user, an option to select a type of test experience; receive, from the user, a selection of the type of test experience, wherein to providing the remote medical diagnostic test to the user is based at least in part on the selection of the type of test experience.

Clause 21. A method for controlling data charges to a user of a medical diagnostic testing service, the method comprising: receiving information related to a user's wireless data plan provider associated with the user; determining if the medical diagnostic testing service can redirect billing by the wireless data plan provider from the user to the medical diagnostic testing service; and based on the determination, providing a different testing experience to the user.

Clause 22. The method of clause 21, wherein the testing experience is modified to reduce an amount of data transferred between a user device and the medical diagnostic testing service.

Clause 23. The method of clause 21, wherein the information related to a user's wireless data plan provider is provided manually by the user.

Clause 24. The method of clause 21, wherein the information related to the user's wireless data plan provider is provided automatically by the wireless data plan provider.

Clause 25. The method of clause 21, wherein the information related to the user's wireless data plan provider is provided automatically by a user device.

Clause 26. A remotely-deployable testing apparatus comprising: a lower outer container portion; a lower inner container portion; an upper outer container portion; an upper inner container portion; one or more fiducial markers; one or more hardware components configured for coupling of the remotely-deployable testing apparatus to a delivery vehicle; and two or more legs coupled to the lower container portion.

Clause 27. The remotely-deployable testing apparatus of clause 26, further comprising: a computing device, wherein the computing device comprises a camera, and wherein the computing device is mounted to the upper inner container portion.

Clause 28. The remotely-deployable testing apparatus of clause 26, further comprising: one or more solar panels; and a charge controller, wherein the charge controller is configured to regulate a voltage, a current, or both provided by the one or more solar panels.

Clause 29. The remotely-deployable testing apparatus of clause 26, further comprising an ultraviolet sterilization compartment.

Clause 30. The remotely-deployable testing apparatus of clause 26, further comprising a receptacle to receive medical waste.

Clause 31. The remotely-deployable testing apparatus of clause 26, further comprising a satellite phone.

Clause 32. The remotely-deployable testing apparatus of clause 26, further comprising wireless networking hardware configured to provide a wireless local area network.

Clause 33. The remotely-deployable testing apparatus of clause 26, further comprising one or more diagnostic tests.

Clause 34. The remotely-deployable testing apparatus of clause 26, further comprising: one or more writable radio frequency identification tags; and an RFID writer, wherein the RFID writer is configured to store one or more diagnostic test results on the one or more writable radio frequency identification tags.

Clause 35. The remotely-deployable testing apparatus of clause 26, further comprising a printer configured to print one or more printable codes, wherein the one or more printable codes comprise one or more diagnostic test results.

Clause 36. The remotely-deployable testing apparatus of clause 27, wherein the computing device is configured to store data relating to one or more diagnostic testing sessions.

What is claimed is:

1. A method for redirecting billing by a wireless data plan provider to a medical diagnostic testing service, the method comprising:
exchanging data between a testing platform and a user device;
generating a message requesting that charges associated with a remote medical diagnostic test be billed to the medical diagnostic testing service; and
transmitting the message from the testing platform to the wireless data plan provider.

2. The method of claim 1, wherein the message is generated by the testing platform.

3. The method of claim 1, wherein the message is generated by the user device.

4. The method of claim 1, wherein the message is transmitted to the wireless data plan provider by the testing platform.

5. The method of claim 1, wherein the message is transmitted to the wireless data plan provider by the user device.

6. The method of claim 1, wherein the message comprises an amount of data used by the remote medical diagnostic test.

7. The method of claim 1, wherein the message comprises an estimated amount of data used by the remote medical diagnostic test.

8. The method of claim 1, wherein the message comprises a start time of the remote medical diagnostic test and an end time of the remote medical diagnostic test.

9. The method of claim 1, wherein the message comprises an amount of data used by a plurality of remote medical diagnostic tests.

10. The method of claim 1, wherein the message comprises a maximum data usage amount.

11. The method of claim 1, wherein the message comprises an estimate of an amount of data used by a plurality of remote medical diagnostic tests.

12. The method of claim 1, wherein the message comprises a plurality of start times and a plurality of end times of a plurality of remote medical diagnostic tests.

13. A system for cost-shifting data used for a remote medical diagnostic test comprising:
a non-transitory computer-readable medium with instructions encoded thereon; and
one or more processors configured to execute the instructions to cause the system to:
receive, from a user device, identifying information indicative of a wireless data plan associated with the user device;
provide the remote medical diagnostic test to a user via a user device;
receive, from the user device, data related to the medical diagnostic test;
determine an indication of an amount of data used for the remote medical diagnostic test;
generate a message to request cost-shifting of the amount of data from the user to a testing platform provider; and
send the message to a provider of the wireless data plan.

14. The system of claim 13, wherein the message comprises the indication of the amount of data used for the remote medical diagnostic test.

15. The system of claim 13, wherein the message comprises identifying information indicative of the wireless data plan.

16. The system of claim 13, wherein the indication of the amount of data used for the remote medical diagnostic test comprises an amount of data used for the remote medical diagnostic test.

17. The system of claim 13, wherein the indication of the amount of data used for the remote medical diagnostic test comprises an estimated amount of data used for the remote medical diagnostic test.

18. The system of claim 13, wherein the indication of the amount of data used for the remote medical diagnostic test comprises a start time and a stop time of the remote medical diagnostic test.

19. The system of claim 13, wherein message comprises a maximum data usage amount.

20. The system of claim 13, wherein the non-transitory computer-readable medium has instructions that encoded thereon that, when executed by the one or more processors, cause the system to:
determine, based on the identifying information, that cost-shifting is not available for the medical diagnostic test; and
provide, to the user, an option to select a type of test experience;
receive, from the user, a selection of the type of test experience,
wherein to providing the remote medical diagnostic test to the user is based at least in part on the selection of the type of test experience.
